SEQUENCE LISTING (32 sequences)

SEQ ID NO: 1 - DNA, 954 bp, Cavia porcellus
agcacctgctgtgggcagcctccctgagaagatgtgtcttctttctctccctctactactctttctccagtacaccagagccaaagctggggaagtcatcggtggcacagagtgcaagccacactcccgcccctacatggcttacctagaaattgtctcttctgagggatatgagaaagactgtagtggtttcttgatacgacggaattttgtgctaacagcagctcactgtgcaggaaggtctctaacagtcaacctaggggtccacaacaaaaaaatgaaagaagacacatggcagaggcttaaggttataaagcaattcctgcatccaaactacaatagctctgtacttctccacgacatcatgttactcaagttggagaagaaagccaacctgaccctagccgtgggcacactccccctcccacctgaatgcaacttcctcacatctggaagaatgtgccgagcagcaggctgggggcgcacgaatgtggaggagcctgcctccgacactcttcaggaggtgaagctgagactcatggatccccaagcctgcaaacacttcccaaactttaaccacaatctccagctgtgtgtgggtaatcctaggaagagaaaatctgtattcaagggagactctggaggtcccctcctgtgtgcagggatagcccagggcattgtatcctatgcacatcggaatgcaaaacctcctgttgtcttcacccgaatctcccattaccggccgtggatcaacaagatcttgaaagcaaattaaccatggagcctcggccagccctgaaaggaaatctggaactggatctgagcaggttttctgtaccacttactctggccctgtctctggttcctgttgaagccagcccatgtccttgagactccagaaagtccctacaactcatagaactctcaataaagcttcagcaaaaatcccaaaaa

SEQ ID NO: 2 - protein, 247 aa, Cavia porcellus
MCLLSLPLLLFLQYTRAKAGEVIGGTECKPHSRPYMAYLEIVSSEGYEKDCSGFLIRRNFVLTAAHCAGRSLTVNLGVHNKKMKEDTWQRLKVIKQFLHPNYNSSVLLHDIMLLKLEKKANLTLAVGTLPLPPECNFLTSGRMCRAAGWGRTNVEEPASDTLQEVKLRLMDPQACKHFPNFNHNLQLCVGNPRKRKSVFKGDSGGPLLCAGIAQGIVSYAHRNAKPPVVFTRISHYRPWINKILKAN

SEQ ID NO: 3 - DNA, 33 bp, Artificial Sequence
  Description of Artificial Sequence Synthetic primer
  modified_base (8)..(9): a, c, g, t, unknown or other
  modified_base (12): a, c, g, t, unknown or other
  modified_base (21): a, c, g, t, unknown or other
garkbyannccncaytcccgnccytatcatggc

SEQ ID NO: 4 - DNA, 31 bp, Artificial Sequence
  Description of Artificial Sequence Synthetic primer
  modified_base (8)..(9): a, c, g, t, unknown or other
  modified_base (12): a, c, g, t, unknown or other
  modified_base (21): a, c, g, t, unknown or other
garkbyannccncaytcccgnccytatcatg

SEQ ID NO: 5 - DNA, 29 bp, Artificial Sequence
  Description of Artificial Sequence Synthetic primer
  modified_base (8)..(9): a, c, g, t, unknown or other
  modified_base (12): a, c, g, t, unknown or other
  modified_base (21): a, c, g, t, unknown or other
garkbyannccncaytcccgnccytatca

SEQ ID NO: 6 - DNA, 31 bp, Artificial Sequence
  Description of Artificial Sequence Synthetic primer
gagtcaaagccacactcccgcccttacatgg

SEQ ID NO: 7 - DNA, 31 bp, Artificial Sequence
  Description of Artificial Sequence Synthetic primer
gagtgcagaccacatgcccgcccctacatgg

SEQ ID NO: 8 - DNA, 29 bp, Artificial Sequence
  Description of Artificial Sequence Synthetic primer
  modified_base (15): a, c, g, t, unknown or other
  modified_base (18): a, c, g, t, unknown or other
kyrcacasdardggnccnccdgagtcycc

SEQ ID NO: 9 - DNA, 26 bp, Artificial Sequence
  Description of Artificial Sequence Synthetic primer
  modified_base (15): a, c, g, t, unknown or other
  modified_base (18): a, c, g, t, unknown or other
kyrcacasdardggnccnccdgagtc

SEQ ID NO: 10 - DNA, 24 bp, Artificial Sequence
  Description of Artificial Sequence Synthetic primer
  modified_base (15): a, c, g, t, unknown or other
  modified_base (18): a, c, g, t, unknown or other
kyrcacasdardggnccnccdgag

SEQ ID NO: 11 - DNA, 29 bp, Artificial Sequence
  Description of Artificial Sequence Synthetic primer
gcacacagaagaggtcccccggagtctcc

SEQ ID NO: 12 - DNA, 29 bp, Artificial Sequence
  Description of Artificial Sequence Synthetic primer
ttacacacaagaggccctccagagtcccc

SEQ ID NO: 13 - DNA, 28 bp, Artificial Sequence
  Description of Artificial Sequence Synthetic primer
gagaaagactgtagtggtttcttgatac

SEQ ID NO: 14 - DNA, 28 bp, Artificial Sequence
  Description of Artificial Sequence Synthetic primer
gtatcaagaaaccactacagtctttctc

SEQ ID NO: 15 - DNA, 24 bp, Artificial Sequence
  Description of Artificial Sequence Synthetic primer
atgctgcttcttcctctccccctg

SEQ ID NO: 16 - DNA, 20 bp, Artificial Sequence
  Description of Artificial Sequence Synthetic primer
ctaacagtcaacctaggggt

SEQ ID NO: 17 - DNA, 20 bp, Artificial Sequence
  Description of Artificial Sequence Synthetic primer
agctcactgtgcaggaaggt

SEQ ID NO: 18 - DNA, 20 bp, Artificial Sequence
  Description of Artificial Sequence Synthetic primer
gacggaattttgtgctaaca

SEQ ID NO: 19 - DNA, 20 bp, Artificial Sequence
  Description of Artificial Sequence Synthetic primer
acccctaggttgactgttag

SEQ ID NO: 20 - DNA, 20 bp, Artificial Sequence
  Description of Artificial Sequence Synthetic primer
accttcctgcacagtgagct

SEQ ID NO: 21 - DNA, 20 bp, Artificial Sequence
  Description of Artificial Sequence Synthetic primer
tgttagcacaaaattccgtc

SEQ ID NO: 22 - protein, 247 aa, Homo sapiens
MLLLPLPLLLFLLCSRAEAGEIIGGTECKPHSRPYMAYLEIVTSNGPSKFCGGFLIRRNFVLTAAHCAGRSITVTLGAHNITEEEDTWQKLEVIKQFRHPKYNTSTLHHDIMLLKLKEKASLTLAVGTLPFPSQFNFVPPGRMCRVAGWGRTGVLKPGSDTLQEVKLRLMDPQACSHFRDFDHNLQLCVGNPRKTKSAFKGDSGGPLLCAGVAQGIVSYGRSDAKPPAVFTRISHYRPWINQILQAN

SEQ ID NO: 23 - DNA, 134 bp, Cavia sp.
  modified_base (77): a, c, g, t, unknown or other
gagtcaaagccacactcccgcccttacatggcttacctagaaattgtctcttctgagggatatgagaaagactgtantggtttcttgatacgacggaattttgtgctaacagcagctcactgtgcaggaaggta

SEQ ID NO: 24 - DNA, 233 bp, Mus sp.
ctgggttccagcaccaaagctggagagatcattggaggcacggagtgcataccacactcccgcccctacatggcctatctggaaattgtcacttctgagaactacctgtcggcctgcagtggcttcctgataagaagaaactttgtgctgactgcagctcactgtgcgggaaggtctataacagtcctcctaggagcccataacaaaacatctaaagaagacacgtggcagaa

SEQ ID NO: 25 - DNA, 116 bp, Cavia sp.
  modified_base (76): a, c, g, t, unknown or other
gagtcaagccacactcccgcccttacatggcttacctagaaattgtctcttctgagggatatgagaaagactgtantggtttcttgatacgacggaattttgtgctaacagcagct

SEQ ID NO: 26 - DNA, 233 bp, Mus sp.
ctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcat

SEQ ID NO: 27 - DNA, 393 bp, Cavia sp.
atgcttcttcctgccctccgtctgctgctctttctcctgggctccagcgcgaggctggcaagatcatcggaggcacagagtgcagaccacatgcccgcccctacatggcctatctggaaattgtcactcccgagaatcacctgtcagcttgcagtggcttcctgataagacgaaactttgtgatgactgctgcgcactgaaccccagctgtgtgtgggcaatcccaagaagattcgaaatgtatacaagggagactctggaggacctctcctgtgtgctgggatagcccaaggcatcgcatcctatgtacttcggaatgcaaagcccccttctgtcttcaccagaatctcccattaccggccctggatcaataagatcttgagggagaattaa

SEQ ID NO: 28 - DNA, 96 bp, Cavia sp.
gagaaagactgtagtggtttcttgatacgacggaattttgtgctaacagcagctcactgtgcaggaaggtctctaacagtcaacctaggggtccat

SEQ ID NO: 29 - DNA, 128 bp, Cavia sp.
cttacctagaaattgtctcttctgagggatatgagaaagactgtagtggtttcttgatacgacggaattttgtgctaacagcagctcactgtgcaggaaggtctctaacagtcaacctaggggtccat

SEQ ID NO: 30 - DNA, 250 bp, Cricetus cricetus
ggagattattggtggtgtggagtcaaagccacactcccgcccttacatggcccatttggagatcgtcactgagagaggctttactgccagctgcggggggttcctcataacgccagaatttgtgatgacggctgcacactgcaaaggaaaagaaattactgtcactcttggagctcatgatgtgagcaaggcagaatccacacagcagaagataaaagtcaaaaaacaaatcgctcacccaaactacagc

SEQ ID NO: 31 - DNA, 250 bp, Cricetus cricetus
caagatcatcggaggcacagagtgcagaccacatgcccgcccctacatggcctatctggaaattgtcactcccgagaatcacctgtcagcttgcagtggcttcctgataagacgaaactttgtgatgactgctgcgcactgtgcaggaaggtctataacagtcctcctaggagcccacaacaaaaaggtaaaagaagacacgtggcagaagcttgaggttgaaaagcaattccctcatccaaaatatgat

SEQ ID NO: 32 - DNA, 98 bp, Cavia sp.
gcagtggcttcctgataagacgaaactttgtgatgactgctgcgcactggcaggaaggtctataacagtcctcctaggagcccacaacaaaaaggtaa

(19) United States
(12) Patent Application Publication
(10) Pub. No.: US 2007/0016970 A1
(43) Pub. Date: Jan. 18, 2007

(54) Guinea pig-chymase

(76) Inventors: Juergen Fingerle, Kandern (DE); Jeremy Beauchamp, Riehen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/484,421

(22) Filed: Jul. 11, 2006

(30) Foreign Application Priority Data
Jul. 13, 2005 (EP) 05106404.6

(51) Int. Cl. A01K 67/027; C07H 21/04; C12P 21/06; C12N 9/64; C12N 5/06

(52) U.S. Cl. 800/14; 435/352; 435/69.1; 435/226; 536/23.2; 435/320.1

(57) ABSTRACT

The present invention relates to the sequence of guinea pig chymase. Since guinea pigs express only one chymase isoform, they are particularly suitable as a model for a method for identifying compounds that modulate chymase.

### PRIORITY TO RELATED APPLICATIONS

This application claims the benefit of European Application No. 05106404.6, filed Jul. 13, 2005, which is hereby incorporated by reference in its entirety.

### FIELD OF THE INVENTION

The present invention relates to guinea pig chymase, the nucleotide sequence that encodes the guinea pig chymase, and methods for identifying compounds that inhibit the activity of chymase and PAOD (peripheral arterial occlusive disease) and counteract asthma, based on the guinea pig chymase.

### BACKGROUND OF THE INVENTION

Chymase is a chymotrypsin-like serine protease which, with the exception of the rat, occurs in the secretory granules of mast cells. In immunology, mast cells are known above all for their role in allergic and fibrotic reactions. If the mast cells are activated, then chymase is released together with histamine and many other mediators via degranulation into the extracellular space. After its release, chymase develops a range of important inflammation-promoting effects. Chymase splits various physiologically and pathologically important proteins, such as e.g. the stem cell factor, big endothelin, TGF beta or apolipoproteins, and is also capable of activating collagenase proteolytically. In addition to these effects, it is assumed that one of the main tasks of human chymase is the production of angiotensin II from angiotensin I. For this reason, it is also speculated that chymase plays an important role in the case of heart failure, high blood pressure, allergy, dermatitis, rheumatic arthritis, asthma and chronic inflammations.

Species such as humans have only one chymase gene, whereas other species have several. It appears as though chymases are very similar to one another if only one is contained in an organism. If several are present, then there appears to be a division of tasks and adaptation to these tasks. Thus animal models with just one chymase are the most suitable for preclinical tests, as humans likewise only have one chymase. In the case of rodents in particular, such as mice and rats, we find numerous chymase genes, and even in hamsters there are two chymase genes.

Therefore, there is a need to find a species of animal that has only a single chymase isoform which at the same time is highly homologous to human chymase, for use in preclinical tests.

### SUMMARY OF THE INVENTION

The present invention accordingly relates to a nucleic acid sequence comprising the sequence of Seq ID No. 1 (FIG. 1). For preference, the nucleic acid sequence is the sequence of Seq ID No. 1. The present invention furthermore relates to a polypeptide sequence comprising the sequence of Seq ID No. 2, wherein the polypeptide sequence is preferably the sequence of Seq ID No. 2 (FIG. 2). The present invention furthermore relates to a nucleic acid sequence comprising a nucleic acid sequence that encodes the polypeptide sequence named above. Furthermore, the present invention relates to an expression vector that comprises the aforementioned nucleic acid sequences. Moreover, the present invention relates to a host cell that comprises the aforementioned expression vector.

Since the guinea pig is the most suitable animal model with a single chymase bioform, the present invention also relates to a guinea pig with somatic cells and germ cells with a mutated chymase gene, wherein at least one allele is functionally interrupted through homologous recombination of the mutated chymase gene, wherein this functional interruption inhibits the expression of the functional chymase gene in the guinea pig cells.

The present invention thus also relates to the following methods for identifying modulators of chymase activity a method for screening compounds that modulate the activity of chymase, comprising peptides: a) bringing a compound into contact with isolated polypeptide sequence of SEQ ID NO. 2; and b) determining the activity of guinea pig chymase; wherein a compound that inhibits or stimulates the activity of the guinea pig chymase is a compound that modulates the activity of the guinea pig chymase.

The present invention also provides a method for identifying compounds that modulate the activity of chymase, comprising a) bringing a compound into contact with the host cell according to claim 5; and b) determining the activity of the guinea pig chymase that is expressed in the host cell; wherein a compound that inhibits or stimulates the activity of the guinea pig chymase is a compound that modulates the activity of the guinea pig chymase.

The present invention also provides a method for identifying compounds that modulate the activity of chymase, comprising a) administering a compound to guinea pigs; and b) determining the activity of the guinea pig chymase; wherein a compound that inhibits or stimulates the activity of the guinea pig chymase is a compound that modulates the activity of the guinea pig chymase.

### BRIEF DESCRIPTION OF THE FIGURES

FIG. 1: Nucleic acid sequence of the guinea pig chymase (Seq ID No. 1)

FIG. 2: Amino acid sequence of the mature guinea pig chymase (Seq ID No. 2)

FIG. 3: Comparison of human chymase and guinea pig chymase

FIG. 4: First sequence of the guinea pig chymase isolated from genomic DNA

FIG. 5: Sequence comparison of mouse chymases with the cloned guinea pig sequence

FIG. 6: Position of the primers for amplification and cloning of the guinea pig chymase cDNA

FIG. 7: Guinea pig sequence from cDNA

FIG. 8: Comparison of the guinea pig chymase with hamster chymase 1 and 2

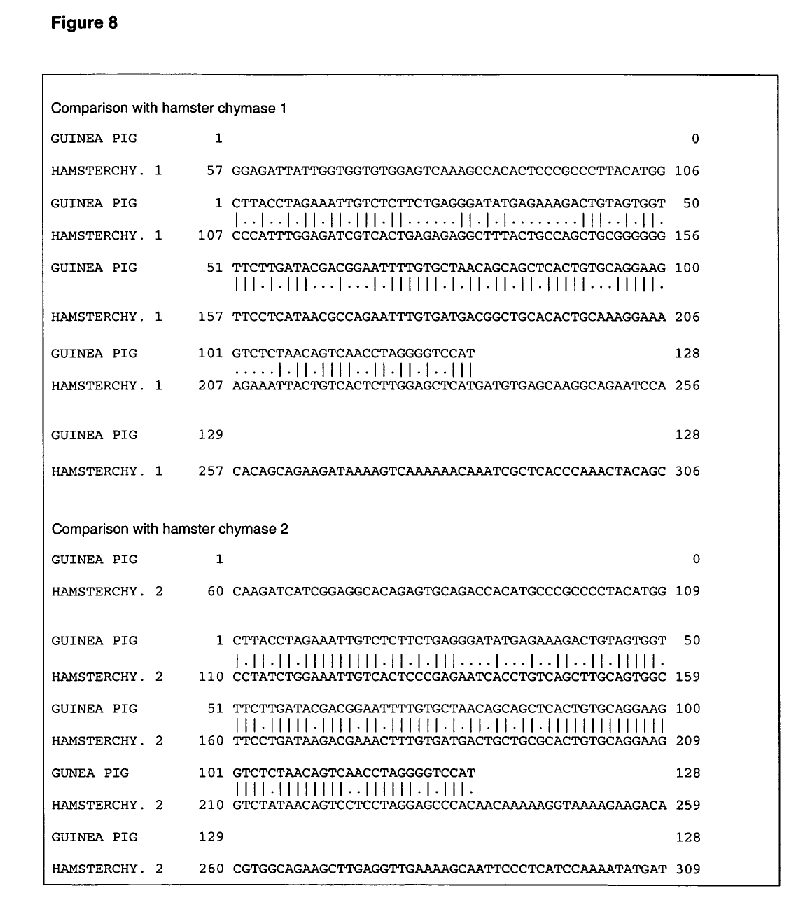

FIG. 9: Position of the primers for cloning of a further sequence of the guinea pig chymase.

### DETAILED DESCRIPTION OF THE INVENTION

The present invention finds a species with only a single chymase isoform which is highly homologous to human chymase through the cloning and provision of guinea pig chymase. For this, both genomic and total RNA were isolated from guinea pig organ samples, in particular from the heart. In order to be able to duplicate chymase or chymase-like sequences in a targeted manner, it was necessary to develop primers with a specificity in respect of these sequences. The primers that were created here are listed in Table 5. With these primers, PCR amplifications starting out from genomic DNA were carried out. In order to enable a statement to be made about the results of the PCRS, the sequences of the plasmid inserts had to be determined. Via a database search, these were then compared with all DNA sequences known to date, and examined for homologies. It was also possible thus to convert the DNA sequences into an amino acid sequence of the resulting protein.

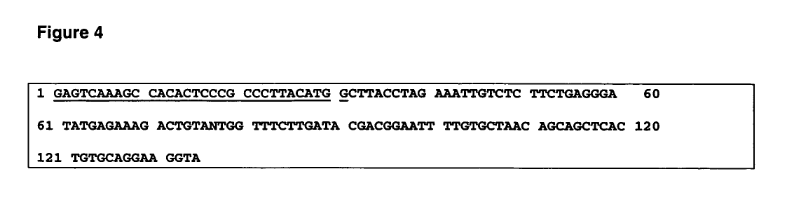

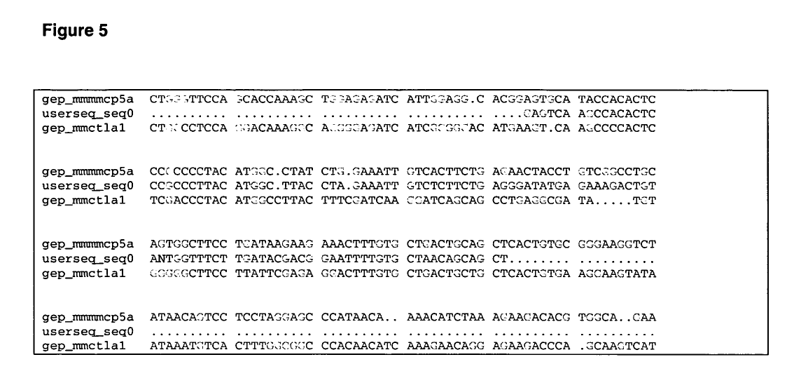

The sequencing of 11 samples yielded the DNA sequences of serine proteases that are already known and are very similar to chymase. However for one sample, for the first time a chymase-like guinea pig part-sequence was found which is shown in FIG. 4. The underlined bases here correspond to the part encoded by the primer. A search in relation to homologies to other DNA sequences showed that this sequence was very similar to DNA molecules already known, such as e.g. the granzyme (gep_mmmmcp5a) or the chymase 1 (gep_mmctala1) of the mouse, but there was no complete agreement. Such a sequence comparison is shown in FIG. 5.

The batches that yielded results contained the primers GP_CHY_{—}12_F and GP_CHY_{—}17_R, and the primers GP_CHY_{—}15_F and GP_CHY_{—}17_R. It would thus appear that of all the primers developed so far, the primer GP_CHY_{—}17_R has the highest specificity in relation to chymase-like sequences in guinea pigs.

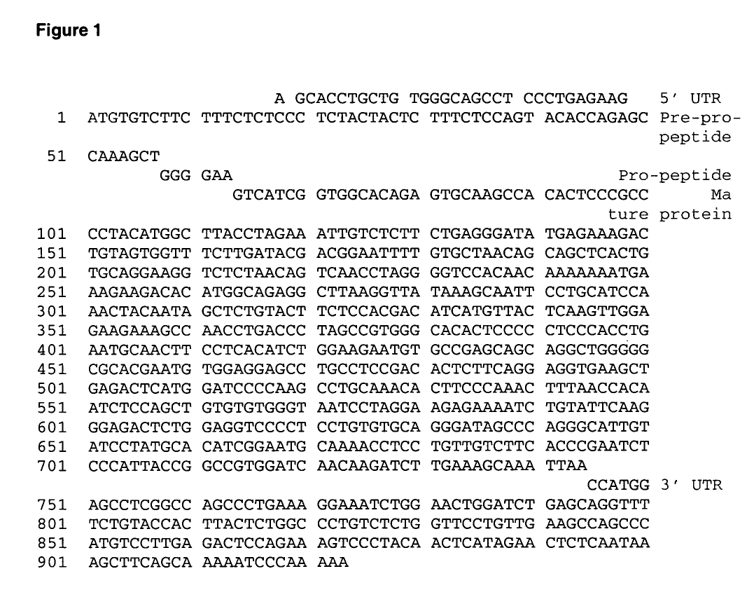

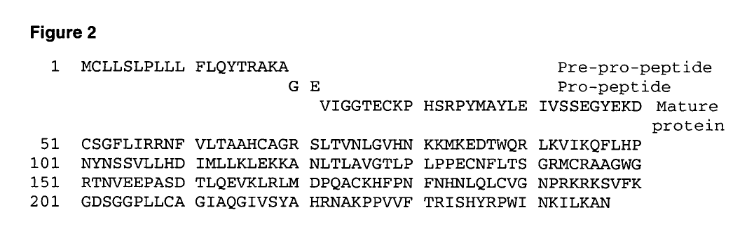

In a next step, on the basis of the sequence isolated from the genomic DNA, the encoded sequence was determined, in that a further fragment of the guinea pig chymase was amplified using specific primers (table 6) from guinea pig cDNA as a template. Starting out from this part-sequence, further primers were selected (table 7), in order to determine a further cDNA sequence of guinea pig chymase. In various reactions, it was possible to determine a further sequence, which is shown in FIG. 1 (Seq ID No. 1). Translation of the nucleic acid sequence in FIG. 1 yields the polypeptide sequence in FIG. 2 (Seq ID No. 2) of the mature chymase, without the signal peptide.

Altogether, 437 sequencings were carried out. In the course of this, guinea pig chymase was found 7 times, granzyme 7 times, but no alternative chymases were found.

The invention accordingly relates to a nucleic acid sequence comprising the sequence of Seq ID No. 1 (FIG. 1). For preference, the nucleic acid sequence is the sequence of Seq ID No. 1. The present invention furthermore relates to a polypeptide sequence comprising the sequence of Seq ID No. 2, wherein the polypeptide sequence is preferably the sequence of Seq ID No. 2 (FIG. 2). The present invention furthermore relates to a nucleic acid sequence comprising a nucleic acid sequence that encodes the polypeptide sequence named above. Furthermore, the present invention relates to an expression vector that comprises the aforementioned nucleic acid sequences. Moreover, the present invention relates to a host cell that comprises the aforementioned expression vector.

The guinea pig chymase polypeptide sequence was compared with known sequences (tables 1 and 2).

TABLE 1
Comparison of known chymase sequences with the mature
guinea pig chymase polypeptide sequence

| Chymase | Abbreviation | Identity (%) | Homology (%) |
|---|---|---|---|
| Human | sw: mcpt1_human | 77.9 | 86.3 |
| Rat 1 | sw: mcpt1_rat | 57.1 | 72.1 |
| Hamster 1 | tr: o08732_mesau | 59.5 | 74.9 |
| Hamster 2 | tr: o70164_mesau | 72.6 | 85.8 |
| Mouse 1 | sw: mcpt5_mouse | 77.0 | 86.3 |

TABLE 2
Comparison with the mature human chymase polypeptide sequence

| Chymase | Abbreviation | Identity (%) | Homology (%) |
|---|---|---|---|
| Guinea pig | xxxx | 77.9 | 86.3 |
| Rat 1 | sw: mcpt1_rat | 57.5 | 72.9 |
| Hamster 1 | tr: o08732_mesau | 59.9 | 74.3 |
| Hamster 2 | tr: o70164_mesau | 70.8 | 81.9 |
| Mouse 1 | sw: mcpt5_mouse | 75.2 | 82.7 |

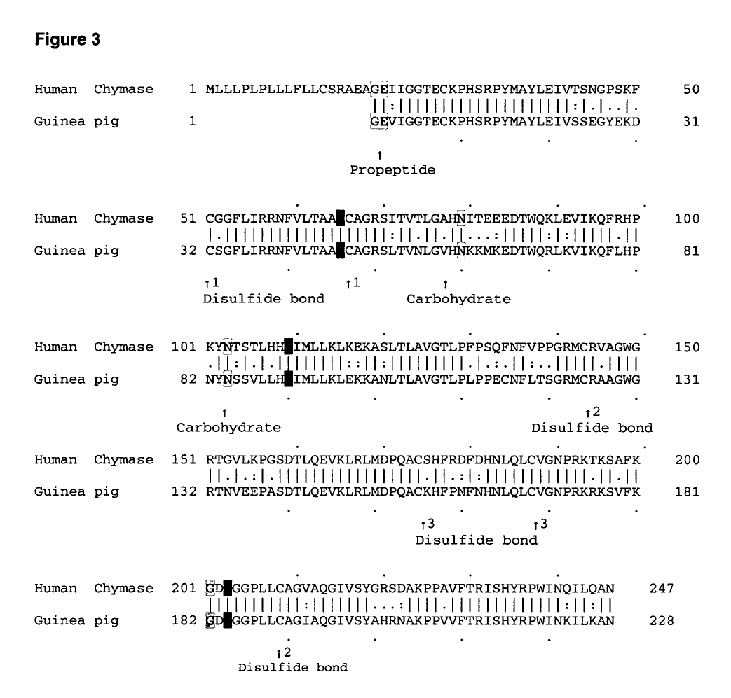

Of the animal species that are compared here, the guinea pig is the one with the highest identity and homology to human chymase (77.9 and 86.3%). The chymases of human, mouse and guinea pig are so similar to one another that all these chymases are α chymases. FIG. 3 shows that the guinea pig chymase contains all the important features of human chymase:
The disulphide bridges are at identical positions
The positions of the active triangle are likewise identical
Both sequences have a glycine at the entrance to pocket 1
The carbohydrates correspond to each other.

Since the guinea pig is the most suitable animal model, the present invention relates to a guinea pig with somatic cells and germ cells with a mutated chymase gene, wherein at least one allele is functionally interrupted through homologous recombination of the mutated chymase gene, wherein this functional interruption inhibits the expression of the functional chymase gene in the guinea pig cells.

The present invention thus also relates to the following methods for identifying modulators of chymase activity and inhibitors of PAOD:

A method is disclosed for screening compounds that modulate the activity of chymase, comprising the steps: a) bringing a compound into contact with isolated guinea pig chymase polypeptide according to claim 2; and b) determining the activity of guinea pig chymase; wherein a compound that inhibits or stimulates the activity of the guinea pig chymase is a compound that modulates the activity of the guinea pig chymase.

Also disclosed is a method for identifying compounds that modulate the activity of chymase, comprising the steps: a) bringing a compound into contact with isolated polypeptide according to claim 2; and b) determining the activity of guinea pig chymase; wherein a compound that inhibits or stimulates the activity of the guinea pig chymase is a compound that modulates the activity of the guinea pig chymase.

A further method is a method for identifying compounds that modulate the activity of chymase, comprising the steps: a) bringing a compound into contact with the host cell according to claim 5; and b) determining the activity of the guinea pig chymase that is expressed in the host cell; wherein a compound that inhibits or stimulates the activity of the guinea pig chymase is a compound that modulates the activity of the guinea pig chymase.

The present invention also discloses a method for identifying compounds that modulate the activity of chymase, comprising the steps: a) administering a compound to guinea pigs; and b) determining the activity of the guinea pig chymase; wherein a compound that inhibits or stimulates the activity of the guinea pig chymase is a compound that modulates the activity of the guinea pig chymase.

Finally, the present invention relates to a method for identifying compounds that inhibit PAOD, comprising the steps: a) administering to guinea pigs a compound that inhibits the guinea pig chymase; b) causing PAOD, and c) determining the extent of PAOD; wherein a compound that inhibits PAOD is a compound that leads to a reduction in the extent of the PAOD that is caused by step b). Preferably, PAOD is caused by administering lauric acid, through damage to a vessel with a balloon catheter or by feeding a diet with a high proportion of fat. Causing PAOD by administering lauric acid is particularly preferred. Another means of causing PAOD that is especially preferred is through damage to a vessel of the guinea pig with a balloon catheter, wherein the vessel is preferably the carotid.

The extent of PAOD is determined using familiar methods. For example, the extent of PAOD is determined by determining the formation of a neointima following a balloon catheter injury e.g. in the carotid of guinea pigs. Another possibility is determining the fatigue of guinea pigs running on a treadmill in the lauric acid model (Kawamura et al., 1985, Arzneimittelforschung 35/7A: 1154-1156).

The present invention also relates to a method for identifying compounds to combat asthma, comprising the steps: a) administering to guinea pigs a compound that inhibits the guinea pig chymase; b) causing asthma; and c) determining the extent of asthma, wherein a compound that inhibits asthma is a compound that leads to a reduction in the extent of the asthma that is caused by step b). In this method, familiar methods are used for inducing asthma and for determining the extent of asthma.

The term “compound”, as used herein, refers to and encompasses small molecules, and specifically and preferably, small molecules analogous to the compounds listed in Table 8. More preferably, the term compound refers to MSK SF2809-V: N-(2-{2-[(4-Hydroxy-1-methyl-2-oxo-1,2-dihydro-quinolin-3-yl)-phenyl-methyl]-1H-indol-3-yl}-ethyl)-acetamide and Toa Eiyo TY51076: 4-(5-Fluoro-3-methyl-benzothiophene-2-sulfonylamino)-3-methanesulfonyl-benzoic acid methyl ester.

The present invention also relates to sequences, methods and guinea pigs as described above, in particular in relation to the following examples.

### EXAMPLES

The following examples are provided for illustrating purposes and are not intended to limit the scope of applicants' invention.

### Example 1

### Cell Disintegration and Obtaining RNA From a Guinea Pig Heart

In order to be able to demonstrate the existence and activity of chymase in guinea pigs, total RNA was isolated. The heart of a guinea pig was used as the organ sample.

### Example 1.1

### Cell Disintegration for Obtaining RNA

Disintegration of the cells took place through the use of so-called Lysing Matrix D Column. For this, 5×10 mg and 5×30 mg of the heart of a guinea pig were filled respectively into tubes with small ceramic balls and overlaid with 300 μl of a mixture of RLT buffer (RNeasy kit) and β-mercaptoethanol (10 μl β-mercaptoethanol per 1 ml of RLT buffer). Subsequently, the tubes were shaken for 2×20 seconds with a FastPrep homogeniser, through which disintegration of the cells was effected through shearing forces. Through centrifugation, the coarsest cell debris was sedimented and the supernatant fluid was transferred into clean tubes.

Subsequently, 590 μl of distilled water and 10 μl 1 mg/ml protein kinase K were added to each tube, the solutions were mixed through inverting and incubated for 10 minutes at 55° C. to break down the proteins. Through centrifugation for 3 minutes at 10,000 rpm and at room temperature, the coarsest cell debris was sedimented and 900 μl of the supernatant fluid was transferred into clean tubes.

### Example 1.2

### RNA Isolation

The isolation of the total RNA from this mixture of proteins and other cell components was carried out using the RNeasy Mini Kit in accordance with the instructions (see Manual RNeasy Kit, protocol for “Isolation of Total RNA from Heart, Muscle and Skin Tissue”).
1. Add 450 μl of 100% EtOH each per batch for denaturing the RNA, mixing the solutions through inverting.
2. Bind the RNA to the silica membrane of the RNeasy columns with collecting vessel by filling the columns with, respectively, 700 μl of this mixture and centrifuging for 30 seconds at 10,000 rpm. and 4° C., discarding the through-flow.
3. Fill the columns with, respectively, 350 μl RW1 buffer and centrifuge for 30 seconds at 10,000 rpm and 4° C. to wash the columns, discarding the through-flow.
4. Transfer the columns to new collecting vessels, filling the columns with, respectively, 500 μl RPE buffer and centrifuging for 30 seconds at 10,000 rpm and 4° C. to wash the column.
5. Fill the columns with a further 500 μl RPE buffer and centrifuge for 2 minutes at 10,000 rpm and 4° C., in order to dry the silica membrane.
6. Transfer the columns to clean 1.5 ml tubes.
7. Add 30 μl RNase free water per column and subsequently elute the RNA through centrifugation for 30 seconds at 13,000 rpm and 4° C.

The concentrations of the various resulting RNA solutions were subsequently determined photometrically, at a wavelength of 260 nm.

### Example 1.3

### Production of cDNA from Guinea Pig RNA

The production of cDNA was carried out by means of the Transcriptor First Strand cDNA Synthesis Kit according to the instructions, as described below. Here, two different batches were prepared, for the use of two different primers.
1. Pipette together:
   8 μl RNA 30.1
   1 μl oligo (dT) primer or 2 μl adapter primer (Invitrogen)
   Fill up with H₂O to 13 μl
2. Incubate the batches for 10 minutes at 65° C. for adding the primers on the RNA.
3. Cool the batches on ice for 2 minutes.
4. Per batch, add:
   4 μl 5× reaction buffer
   0.5 μl RNase Inhibitor
   2 μl dNTPs
   0.5 μl reverse transcriptase
5. Incubate the batches for 1 hour at 50° C. for the procedure of the reverse transcriptase reaction.
6. Incubate for 5 minutes at 85° C. to inactivate the reverse transcriptase.
7. Cool the batches on ice for 2 minutes.

To remove low-molecular fragments and unused constituents of the RT-PCR, the different batches were purified by means of the QIAquick PCR Purification Kit according to the instructions for microcentrifuges, as follows:
1. Add 250 μl of PB buffer per RT-PCR batch, mixing the solutions.
2. Fill the QIAquick columns with collecting containers with one batch each and centrifuge for 1 minute at 13,000 rpm and 4° C. to bind the DNA to the silica membrane of the column, discarding the through-flow.
3. To wash the columns, add in each case 750 μl PE buffer and centrifuge for 1 minute at 13,000 rpm and 4° C., discarding the through-flow.
4. Centrifuge the columns for 1 minute at 13,000 rpm and 4° C. to dry the silica membrane.
5. Transfer the columns into clean 1.5 μl tubes.
6. Add 30 μl EB buffer per column, incubate for 1 minute at room temperature and subsequently elute the cDNA through centrifugation for 1 minute at 13,000 rpm and 4° C.

### Example 2

### Cell Disintegration and Obtaining Genome DNA from Guinea Pig Heart

Another possibility for demonstrating chymase in guinea pigs was the use of genome DNA. This method was used since it could not be assumed with certainty that the gene that was sought was in fact expressed.

### Example 2.1

### Cell Disintegration

To obtain genome DNA, as in the case of RNA, disintegration of the cells was necessary. Once again, this was carried out via the aforementioned use of Lysing Matrix D columns. For this, 2×100 of the heart of a guinea pig were filled respectively into tubes with small ceramic balls and overlaid with 1.2 μl of digestion buffer. The composition of this buffer is shown in table 2-13.

TABLE 3
Digestion buffer

| Component | Concentration | |
|---|---|---|
| NaCl | 100 | mM |
| Tris-HCl pH 8.0 | 10 | mM |
| EDTA pH 8.0 | 25 | mM |
| SDS | 0.5% | (w/v) |
| Protein kinase K | 0.1 | mg/ml |

Subsequently, the tubes were shaken for 2×20 seconds with a FastPrep homogeniser, through which disintegration of the cells was effected through shearing forces.

### Example 2.2

### Isolation of the DNA via Phenol Extraction and Ethanol Precipitation

The separation of proteins and DNA took place via phenol extraction by means of Phase Lock Gel according to the instructions, as follows.
1. Centrifuge the gel down in the vessel (2.0 ml) at 13,000 rpm for 20-30 seconds.
2. Add 100-750 μl disintegrated cells per vessel.
3. Add the same volume of organic solvent, in this case phenol:chloroform:isoamyl alcohol (25:24:1).
4. Mix for 10 minutes through occasional inversion (no vortexes!)
5. Centrifuge for phase separation at 13,000 rpm for 5 minutes.
6. Take off the supernatant fluid and process further.

Subsequently, the DNA was cleansed and concentrated via ethanol precipitation, as detailed below.
1. Transfer upper phase after phenol extraction into a new cup and add 1/10 of its own volume of 3 M sodium acetate (NaAc) pH 5.0 and 2.5 of its own volume of 100% EtOH.
2. Fish out the denatured DNA molecules with a sterile inoculating loop and transfer into a new cup.
3. Renature the DNA in 1.5 ml 10 mM tris-HCl pH 7.5 and repeat the addition of EtOH NaAc.
4. Centrifuge for 15 minutes at 11,200 rpm and 4° C.
5. Remove the supernatant fluid and resuspend the pellet in 1.5 ml 75% EtOH.
6. For salt removal, invert the suspension for 10 minutes.
7. Centrifuge for 10 minutes at 11,200 rpm and 4° C., remove supernatant fluid and air-dry the pellet for 10-15 minutes.
8. Dissolve the pellet in 1.5 ml 10 mM tris-HCl pH 7.5.

### Example 3

### Search for Chymase Coding Sequences in Genome DNA

In order to be able to identify chymase sequences where the respective gene is inactive, it is necessary to multiply these sequences from genome DNA templates by means of a PCR. In this part-experiment, different degenerated primers which have a specificity for guinea pig chymase were developed and used for this purpose.

### Example 3.1

### Development of Primers for Guinea Pig Chymase

It was presumed that chymase showed great homology in all animal models. Thus starting from the sequence for hamster chymase 1, which was already known, a database search was started for other known DNA sequences with as much in common as possible. The positive sequences of this search, which included e.g. many serine proteases and chymases of mice or rats, were then compared with one another, and from the regions with the widest variations between the individual sequences, primers were developed of which it could be assumed that they would bind chymase or chymase-like sequences. Within the individual primers, to some extent variations were allowed, in order to obtain a greater selection of sequences. These variations were produced by allowing several different bases at individual positions of the sequences. Such primers are termed degenerated. The variations were expressed via the generally applicable letter codes shown in table 4; production of these primers was undertaken by a contract firm.

TABLE 4
Letter codes

| Letter | Possible bases |
|---|---|
| B | C, G or T |
| D | A, G or T |
| H | A, C or T |
| K | G or T |
| M | A or C |
| N | A, C, G or T |
| R | A or G |
| S | C or G |
| V | A, C or G |
| W | A or T |
| Y | C or T |

TABLE 5

Primers developed

Transcription

| | | |
|---|---|---|
| Primer name | Sequence | direction |

| | | |
|---|---|---|
| GP_CHY_12_F | GARKBYANNCCNCAYTCCCGNCCYTA | Forward |
| | TCATGGC | (seq ID 3) |
| GP_CHY_13_F | GARKBYANNCCNCAYTCCCGNCCYTA | Forward |
| | TCATG | (seq ID 4) |
| GP_CHY_14_F | GARKBYANNCCNCAYTCCCGNCCYTA | Forward |
| | TCA | (seq ID 5) |
| GP_CHY_15_F | GAGTCAAAGCCACACTCCCGCCCTTA | Forward |
| | CATGG | (seq ID 6) |
| GP_CHY_16_F | GAGTGCAGACCACATGCCCGCCCCTA | Forward |
| | CATGG | (seq ID 7) |
| GP_CHY_17_R | KYRCACASDARDGGNCCNCCDGAGTC | Reverse |
| | YCC | (seq ID 8) |
| GP_CHY_18_R | KYRCACASDARDGGNCCNCCDGAGTC | Reverse |
| | | (seq ID 9) |
| GP_CHY_19_R | KYRCACASDARDGGNCCNCCDGAG | Reverse |
| | | (seq ID 10) |
| GP_CHY_20_R | GCACACAGAAGAGGTCCCCCGGAGTC | Reverse |
| | TCC | (seq ID 11) |
| GP_CHY_21_R | TTACACACAAGAGGCCCTCCAGAGTC | Reverse |
| | CCC | (seq ID 12) |

### Example 3.2

### PCR with the Primers that were Produced

Through the use of the degenerated primers, the attempt was to be made to amplify chymase or chymase-like sequences. For this purpose, each of the F primers was combined with each R primer.

The PCR batches were composed as follows:
1 μl template (genome DNA)
1 μl 1^{st} primer (GP_CHY_{—}12_F to 16_F)
1 μl 2^{nd} primer (GP_CHY_{—}17_F to 21_R)
20 μl HotStarTaq Master Mix
17 μl H₂O

The PCR proceeded according to the following program:

| | | | |
|---|---|---|---|
| 95° C. | 10 minutes | | |
| 95° C. | 30 seconds | | |
| 55° C. | 30 seconds | {close oversize bracket} | 30 cycles |
| 72° C. | 2 minutes 30 seconds | | |
| 72° C. | 10 minutes | | |
| 4° C. | storage | | |

The PCR products were analysed with a 1% agarose/EtBr gel.

### Example 4

### Multiplication of the PCR Products that have been Obtained

In order to be have sufficient material in respect of PCR products that have been obtained available for further investigations, these were amplified by means of transformation and via the cultivation of bacterial cultures.

### Example 4.1

### Purification of the PCR Batches

In order to remove low-molecular fragments and other interference substances for the later transformation, selected PCR batches were purified with the QIAquick PCR Purification Kit according to the instructions, as described above.

### Example 4.2

### Transformation with the TOPO TA Cloning Kit

Since several products or low-molecular interference fragments frequently occur in PCR batches, in most cases direct sequencing is not possible. For this reason, for separation and amplification, the PCR products were incorporated into bacterial vectors and the plasmids were subsequently introduced into competent cells. This was carried out by means of the TOPO TA Cloning Kit, as described below.
1. Mix 4 μl PCR batch, 1 μl saline solution and 1 μl TOPO vector. Incubate the batches for 5 minutes at room temperature.
2. Add 2.5 μl of this mixture to a tube of competent TOP 10 F′ cells which were thawed on ice.
3. Incubate the cells for 5 minutes on ice, to take the plasmids into the cells.
4. Heat shock for 30 seconds at 42° C. to stop DNA uptake by the cells, and abrupt cooling off on ice for 2 minutes.
5. Add 300 μl LB medium per bacteria tube and incubate for 1 hour at 37° C. on the shaker.
6. Per batch, inoculate two LB/amp plates via plating out the bacteria, and incubate overnight at 37° C.

### Example 4.3

### Multiplication of PCR Products in Culture and Isolation of Plasmids

In order to obtain cultures that multiply only a particular plasmid, per batch 8 individual colonies were taken with sterile pipette tips from the plates obtained in chapter 2.7.2, and used for inoculating in each case 1.5 ml LB/amp medium. These were cultivated overnight at 37° C. on the shaker.

After completion of cultivation, 1 ml per culture was taken, transferred into a clean 1.5 ml tube or—depending on the number of samples—into a 96-well plate. The cells were then centrifuged off for 15 minutes at 4° C. and 3,000 rpm, and the supernatant fluid was removed. Subsequently, plasmid isolation was carried out with the QIAprep 8 Turbo Miniprep Kit according to the instructions, using the QIAvac 6S apparatus as described below.
1. Resuspend the bacterial pellet in 250 μl P1 buffer through vortexing.
2. Add 250 μl P2 buffer per tube for lysis of the bacteria, and mix through careful inverting for 4 minutes.
3. Add 350 μl N3 buffer to precipitate the proteins and mix through careful inverting for 4 minutes.
4. Set up the QIAvac 6S apparatus.
5. Transfer the supernatant fluids into the turbo filter strip in the upper plate and apply the vacuum until all the samples have run into the columns underneath.
6. Remove the turbo filter strip and place the column strip with samples in the upper plate, replace the strip holder in the base plate by the collecting trough.
7. Apply the vacuum until all the liquid has passed through the column.
8. Add 1 ml PB buffer to wash the columns and apply the vacuum until the liquid has passed through the column.
9. Add 1 ml PE buffer to wash the columns and apply the vacuum until the liquid has passed through the column. Repeat this step once.
10. Apply the maximum vacuum for 5 minutes to dry the column membranes, then remove the entire upper plate and dry the column outlets with a highly absorbent paper towel.
11. Replace the collector trough with a 96-well plate, which has been raised through microtube stands placed underneath.
12. Fit the upper plate into the apparatus and add 100 μl EB buffer per column, incubate for 1 minute at room temperature.
13. To elute the samples, apply maximum vacuum for 5 minutes.

### Example 4.4

### Restriction Digestion of the Isolated Plasmids

In order to separate the inserts from the bacterial vector, and to check them, restriction digestion was carried out according to the following pipetting schema:
10 μl plasmid
1.5 μl 10×EcoR I buffer
0.15 μl BSA
1 μl EcoR I
2.5 μl H₂O

The restriction digestion was incubated for 1 hour at 37° C., and subsequently checked with a 1% agarose/EtBr gel.

### Example 5

### Sequencing the Multiplied PCR Products

In order to be able to examine the PCR products which had been amplified by means of bacterial plasmids for chymase sequences, their sequences were now established.

### Example 5.1

### Sequencing of Selected Plasmids

Due to the restriction digestion, it was possible to identify batches with different inserts, so that we did not sequence identical inserts several times over. For the sequencing, it was first of all necessary to carry out a chain termination synthesis with fluorescence-marked ddNTPs. These were available together with the reaction buffer and the normal dNTPS in the form of a ready mix called Big Dye (commercially attainable, Applied BioSystems (ABI), Switzerland Applied Biosystems, Rotkreuz Branch, Grundstrasse 10, CH-6343 Rotkreuz, Switzerland).

The following volumes were used for the sequencing:
5 μl plasmid solution
5 μl Big Dye
1 μl primer M13-R

The primer M13-R was used for the sequencings, since this binds immediately outside the multicloning site, and thus each insert could be sequenced completely independently of its type.

The chain termination synthesis proceeded according to the following program:

| | | | |
|---|---|---|---|
| 95° C. | 2 minutes | | |
| 95° C. | 30 seconds | | |
| 45° C. | 30 seconds | {close oversize bracket} | 40 cycles |
| 60° C. | 4 minutes | | |
| 4° C. | storage | | |

Subsequently the batches were freed of the remaining fluorescent dyes by means of the Dye Ex 2.0 kit, according to the instructions, as these would interfere with the sequencing.
1. Vortex the Dye Ex columns with gel material for 30 seconds.
2. Open the closure by a ¼ turn of the lid.
3. Break off the closure of the column outlet by bending (not twisting).
4. Place the columns in the collecting container and centrifuge for 3 minutes at 3,000 rpm.
5. Transfer the columns into clean 1.5 ml tubes.
6. Apply the samples to the centre of the oblique gel surface without touching it with the tip of the pipette.
7. Centrifuge the columns for 3 minutes at 3,000 rpm to elute the samples.

The purified samples were then sequenced automatically using a sequencer, and the base sequences that were obtained were compared via a database search with all known DNA molecules. Furthermore, likewise via a database search, from the DNA sequences that were obtained, the corresponding amino acid sequences of the resulting proteins were determined.

### Example 6

### Search for Chymase Coding Sequences in RNA with Specific Primers

### Example 6.1

### Production of Guinea Pig Sequence Specific Primers

With the aid of the first DNA part-sequence from guinea pigs, primers specific to this were to be developed. For this, those parts of the guinea pig sequence were selected that differed most clearly from the sequences of the comparison DNA of hamsters and humans, and primers of a 30 base length were developed. These primers were then produced by a contract firm.

TABLE 6

Specific primers for guinea pig chymase

Transcription

| | | |
|---|---|---|
| Primer name | Sequence | direction |

| | | |
|---|---|---|
| CHYGP_22_F | GAGAAAGACTGTAGTGGTTTCTTGAT | Forward |
| | AC | (seq ID 13) |
| CHYGP_23_R | GTATCAAGAAACCACTACAGTCTTTC | Reverse |
| | TC | (seq ID 14) |
| CHYGP_24_F | ATGCTGCTTCTTCCTCTCCCCCTG | Forward |
| | | (seq ID 15) |

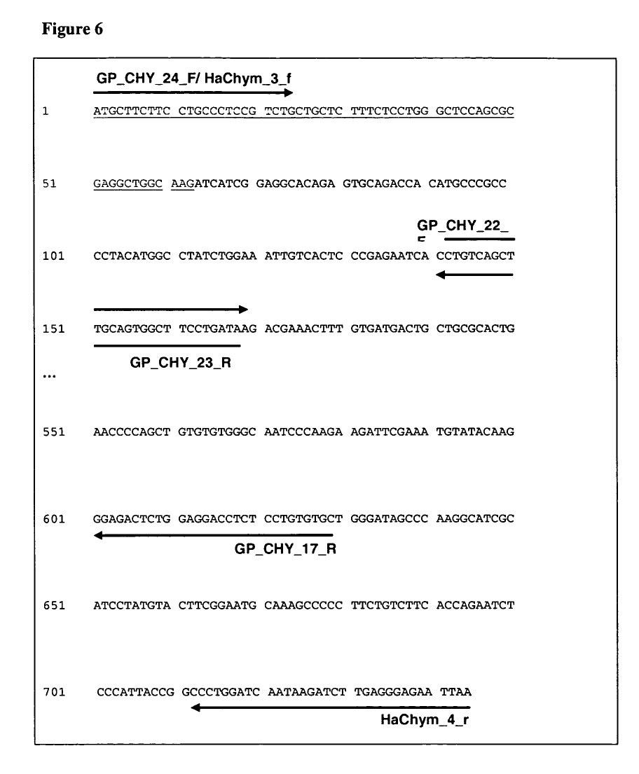

The primers used for the subsequent PCRs are shown in FIG. 6.

### Example 6.2

### PCR with Different Primers

The task was once more to carry out a wide-ranging search for chymase sequences in guinea pig RNA. For this, the most varied primers that had been developed or used to date were combined with one another; amongst other things, hamster chymase primer was used again, and also the oligo (dT) primer as well as the universal adapter primer (UAP, Invitrogen). The cDNA solutions that were created with the primers oligo (dT) and adapter primer were used as templates. For the batches F and J, only the adapter primer template was used, and for the batches G and K the oligo (dT) primer template was used. All other batches were produced for both templates. In addition, batches G and K were exposed to an annealing temperature of 45° C., whereas for the others this was 58° C.

Batches were prepared with these primer combinations:
A: HaChym_{—}3_f+GP_CHY_{—}17_R
B: GP_CHY_{—}12_F+HaChym_{—}4_r
C: GP_CHY_{—}15_F+HaChym_{—}4_r
D: GP_CHY_{—}22_F+GP_CHY_{—}17_R
E: GP_CHY_{—}22_F+HaChym_{—}4_r
F: GP_CHY_{—}22_F+UAP Primer
G: GP_CHY_{—}22_F+Oligo (dT) Primer
H: GP_CHY_{—}24_F+GP_CHY_{—}17_R
I: GP_CHY_{—}24_F+HaChym_{—}4_r
J: GP_CHY_{—}24_F+UAP Primer
K: GP_CHY_{—}24_F+Oligo (dT) Primer
L: GP_CHY_{—}24_F+GP_CHY_{—}23_R
M: HaChym_{—}3_f+GP_CHY_{—}23_R

The batches were composed as follows:
1 μl cDNA template
1 μl 1^{st} primer
1 μl 2^{nd} primer
20 μl HotStarTaq Master Mix
17 μl H₂O

The PCR proceeded according to the following program:

| | | | |
|---|---|---|---|
| 95° C. | 10 minutes | | |
| 95° C. | 30 seconds | | |
| 58 or 45° C. | 30 seconds | {close oversize bracket} | 30 cycles |
| 72° C. | 3 minutes | | |
| 72° C. | 10 minutes | | |
| 4° C. | storage | | |

The PCR batches were checked with a 1% agarose/EtBr gel.

### Example 7

### Investigation and Multiplication of Selected PCR Products

In order to have sufficient material available for investigation, selected promising PCR products were to be multiplied via transformation and bacterial cultures, and subsequently investigated for possible guinea pig sequences. For this, the batches F_{AP}, J_{AP}, H_{dI}, H_{AP}, I_{dI}, I_{AP}, L_{dI} and L_{AP} were selected.

### Example 7.1

### Amplification Through TOPO TA Cloning and Plasmid Isolation

The PCR products that were obtained and were purified by means of the QIAquick Purification Kit were once again incorporated into bacterial vectors by means of the TOPO TA cloning kit, and introduced into competent cells by means of transformation. The resulting LB/amp plates were incubated overnight at 37° C. in the incubator.

### Example 7.2

### Colony PCR and Bacterial Cultures

For each batch, 6 individual colonies were taken from the plates resulting from item 2.10.1, by means of sterile pipette tips. These were first of all dipped several times into a PCR batch. In the course of this, bacteria were released which in the subsequent PCR were killed off and disintegrated in the first 95° C. step, so that the plasmids contained in them could serve as templates.

The batches that were produced were composed as follows:
1 μl primer M13-F
1 μl primer M13-R
5 μl HotStarTaq Master Mix
3 μl H₂O

The PCR proceeded according to the following program:

| | | | |
|---|---|---|---|
| 95° C. | 15 minutes | | |
| 95° C. | 30 seconds | | |
| 45° C. | 30 seconds | {close oversize bracket} | 30 cycles |
| 72° C. | 1 minute 30 seconds | | |
| 72° C. | 5 minutes | | |
| 4° C. | storage | | |

Furthermore, the tips were then used as usual to inoculate in each case 1.5 ml LB/amp medium in a 48-well plate.

Cultivation of the liquid cultures took place overnight at 37° C. on the shaker. Subsequently, the plasmids were isolated as described above, using the QIAprep 8 Turbo Miniprep Kit.

Checking the Colony PCR

Through the colony PCR, carried out in parallel with the cultivation, the intention was to make it possible at an early stage to make a statement about the inserts contained in the liquid cultures, and thus to be able to make a pre-selection of the cultures to be processed further. The results of the colony PCR were investigated by means of a 1% agarose/EtBr gel.

### Example 7.3

### Sequencing of Selected Plasmids

Plasmids were isolated by means of the QIAprep 8 Turbo Miniprep Kit.

For the sequencing of the plasmids from the batches listed above, it was necessary to carry out chain termination synthesis with fluorescent-marked ddNTPs beforehand.

The batches are composed as follows:
5 μl plasmid solution
5 μl Big Dye
1 μl primer M13-R

The sequencing PCR proceeded according to the following program:

| | | | |
|---|---|---|---|
| 95° C. | 1 minute | | |
| 95° C. | 30 seconds | | |
| 45° C. | 30 seconds | {close oversize bracket} | 40 cycles |
| 60° C. | 4 minutes | | |
| 4° C. | storage | | |

Subsequently, the batches were freed of the remaining fluorescent dye by means of the Dye Ex 2.0 kit, as these would interfere with the sequencing. The purified samples were then sequenced automatically by means of a sequencer, and the sequences that were obtained were compared via a database search with all known DNA molecules. Furthermore, likewise via a database search, from the DNA sequences that were obtained, the corresponding amino acid sequences of the resulting proteins were determined.

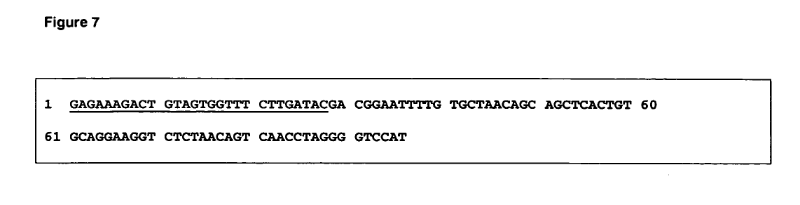

The guinea pig sequence thus obtained is shown in FIG. 7.

### Example 8

### Completion of the Sequence of Guinea Pig Chymase

The sequences of putative guinea pig chymase that had been discovered and sequenced so far corresponded, in the case of the already fully known sequence for hamster chymase, roughly to the nucleic acid sequence that codes mature chymase. The task was now to determine, starting out from this part sequence, the rest of the chymase in the 5′ direction. This was to be carried out via a so-called 5′ RACE.

### Example 8.1

### Developing New Primers

New specific primers were selected on the basis of the newly determined sequence (table 7). These primers were produced by a contract firm. (Operon Biotechnologies GmbH, Nattermannallee 1, D-50829 Cologne, Germany).

TABLE 7

Primers developed

Transcription

| | | |
|---|---|---|
| Primer name | Sequence | direction |

| | | |
|---|---|---|
| GP_CHY_25_F | CTAACAGTCAACCTAGGGGT | Forward |
| | | (seq ID 16) |
| GP_CHY_26_F | AGCTCACTGTGCAGGAAGGT | Forward |
| | | (seq ID 17) |
| GP_CHY_27_F | GACGGAATTTTGTGCTAACA | Forward |
| | | (seq ID 18) |
| GP_CHY_28_R | ACCCCTAGGTTGACTGTTAG | Reverse |
| | | (seq ID 19) |
| GP_CHY_29_R | ACCTTCCTGCACAGTGAGCT | Reverse |
| | | (seq ID 20) |
| GP_CHY_30_R | TGTTAGCACAAAATTCCGTC | Reverse |
| | | (seq ID 21) |

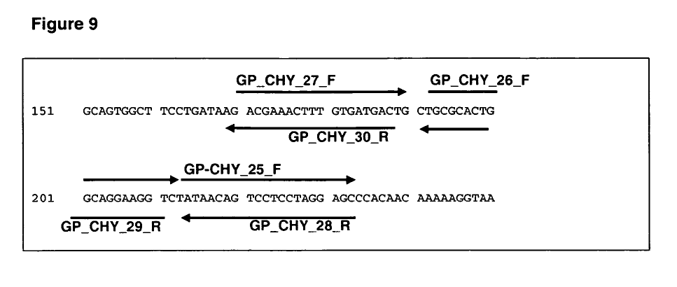

The position of the primers is shown in FIG. 9.

### Example 8.2

### Production of Different cDNA Templates

In order to achieve the best possible results and to be able to exclude any degeneration of the templates, 4 different cDNA templates were produced through the use of two different kits, as described below.

Transcriptor First Strand cDNA Synthesis Kit
1. Pipette together 2×:
2 μl RNA 30/1 (see chapter 3.1)
2 μl random primer (cDNA 1) or adapter primer (cDNA 2)
9 μl H₂O
1. Incubate the mixture for 10 minutes at 65° C. for adding the primers.
2. Cool on ice for 2 minutes.
3. Per batch, add:
4 μl reaction buffer
0.5 μl RNase Inhibitor
2 μl dNTPs
0.5 μl reverse transcriptase
4. Incubate the batches for 1 hour at 50° C. for the procedure of the reverse transcriptase reaction.
5. Inactivate the reverse transcriptase for 5 minutes at 85° C.
6. Cool the batches on ice.

5′ RACE Kit for Rapid Amplification of cDNA Ends
1. Producing cDNA:
   Pipette together 2×:
0.25 μl 20 μM GP_CHY_{—}28_R
8 μl RNA 30/3 and 30/4 (see chapter 3.1)
7.25 μl H₂O
   Incubate for 10 minutes at 70° C. for adding the primers.
   Cool on ice for 1 minute.
   Per batch, add:
2.5 μl 10×PCR buffer
2.5 μl MgCl₂ solution
1 μl dNTPs
2.5 μl DTT
   Pre-heat for 1 minute at 42° C.
   Add 1 μl SS II reverse transcriptase per batch.
   Incubate for 50 minutes at 42° C. for the procedure of the reverse transcriptase reaction.
   Inactivate the reverse transcriptase for 15 minutes at 70° C.
   Incubate for 1 minute at 37° C.
   Add 1 μl RNase mix per batch.
   Incubate for 30 minutes at 37° C. to break down the RNA
   Store batches on ice.
2. S.N.A.P. purification of the batches:
   Add 120 μl binding buffer per sample
   Transfer the samples into the S.N.A.P. columns, centrifuge for 20 seconds at 13,000 rpm and 4° C.
   For safety, store the through-flow on ice
   Add 400 μl cold 1× washing buffer per column, centrifuge off, and discard the through-flow. Repeat this procedure 3×.
   Wash the columns 2× with 400 μl cold 70% EtOH, and discard the through-flow.
   Centrifuge for 1 minute at 13,000 rpm and 4° C. to dry the membrane.
   Transfer the columns into fresh tubes, add in each case 50 μl water that has been pre-heated to 65° C.
   Centrifuge for 20 seconds at 13,000 rpm to elute the cDNA (cDNA 3).
3. Appending a polycytosine tail to a part of the cDNA 3 via dC tailing:
   Pipette together:
6.5 μl DEPC treated water
5 μl 5× tailing buffer
2.5 μl 2 mM dCTP
10 μl purified cDNA
   Incubate for 2 minutes at 94° C.
   Cool on ice for 1 minute.
   Add 1 μl TdT (name) per batch.
   For the procedure of the dC tailing, incubate for 10 minutes at 37° C.
   Inactivate the TdT for 10 minutes at 65° C.
   Cool the batches on ice (cDNA 4).

### Example 8.3

### PCR to Multiply the Newly Obtained cDNA Fragments

The quantities of cDNA that were obtained in Example 8.2 above were multiplied via the PCR described below.

For the PCR, the volumes listed here were pipetted together on ice:

| | |
|---|---|
| 34.4 μl | H₂O |
| 5 μl | Expand High Fidelity PCR buffer + MgCl₂ |
| 1 μl | dNTPs |
| 2 μl | 1^{st} primer |
| 2 μl | 2^{nd} primer |
| 5 μl | cDNA |
| 0.6 μl | Taq polymerase (3.5 U/μl) |

These primer combinations were used here:
cDNA 1: GP_CHY_{—}17_R+27_F
cDNA 2: GP_CHY_{—}17_R+27_F and UAP+GP_CHY_{—}27_F
cDNA 3: GP_CHY_{—}24_F+29_R
cDNA 4: GP_CHY_{—}24_F+29_R and UAP+GP_CHY_{—}29_R

For the batch with the cDNA 2, in which the UAP was used, the 3′ RACE took place, and for the batch with cDNA 4 correspondingly the 5′ RACE.

The PCR proceeded according to the following program:

| | | | |
|---|---|---|---|
| 95° C. | 10 minutes | | |
| 95° C. | 30 seconds | | |
| 55 | 30 seconds | {close oversize bracket} | 35 cycles |
| 72° C. | 3 minutes | | |
| 72° C. | 10 minutes | | |
| 4° C. | storage | | |

The samples were then checked with a 1% agarose/EtBr gel.

### Example 8.4

### Nested Amplification

Since simple PCR was mostly insufficient for producing visible amounts in this obtaining of cDNA, a further PCR was carried out, which in this connection was referred to as “nested amplification”.

For this, the following volumes were pipetted together:

| | |
|---|---|
| 36.4 μl | H₂O |
| 5 μl | Expand High Fidelity PCR buffer + MgCl₂ |
| 1 μl | dNTPs |
| 1 μl | 1^{st} primer |
| 1 μl | 2^{nd} primer |
| 5 μl | cDNA from PCR 1 (diluted 1:100) |
| 0.6 μl | Taq polymerase (3.5 U/μl) |

These primer combinations were used here:
cDNA 1: GP_CHY_{—}17_R+26_F (1) and GP_CHY_{—}17_R+25_F (2)
cDNA 2-17: GP_CHY_{—}17_R+26_F (3) and GP_CHY_{—}17_R+25_F (4)
cDNA 2-UAP: UAP+GP_CHY_{—}26_F (5) and UAP+GP_CHY_{—}25_F (6)
cDNA 3: GP_CHY_{—}24_F+30_R (7)
cDNA 4-24: GP_CHY_{—}24_F+30_R (8)
cDNA 4-UAP: GP_CHY_{—}24_F+30_R (9) and UAP+30_R (10)

The amplification products were subsequently checked with a 1% agarose/EtBr gel.

The products of the nested amplification are subsequently cloned, as described above, multiplied and sequenced.

### Example 9

### Expression of Recombinant Guinea Pig Chymase

Recombinant guinea pig chymase is expressed by means of the cloned sequence (Seq ID No. 1) in a suitable expression system, e.g. Pichia pastoris, and purified (Nakabuko et al, 2000, Yeast 16(4), 315-23; Takai et al., 1997, Clin. Chim. Acta 265, 13-20; Schechter et al., 1993, J. Biol. Chem. 268, 23626-23633), which are hereby incorporated by reference in their entirety.

### Example 9.1

### Expression & Purification of Guinea Pig Chymase

The recombinant guinea pig chymase was expressed in BL21(DE3) cells as an N-terminal fusion protein with a 26-amino acid polypeptide carrying a 6×His tag and a enterokinase cleavage site. Since the protein was expressed forming insoluble inclusion bodies it had to be renatured. Isolated and purified inclusion bodies were modified under denatured conditions with glutathione and then dissolved in 6 M guanidine HCl, 20 mM EDTA pH 4.5. Refolding was done by a 1:100 dilution of the protein solution into a large volume of refolding buffer: 50 mM Tris/HCl pH 8.0, 750 mM arginine, 1 mM EDTA, 2 mM cysteine. Alkaline pH was chosen to promote thiolate anion formation and hence disulphide exchange. The preparation was kept at 4° C. for two days. Due to the high ionic strength of the refolding solution, the whole preparation had to be concentrated by ultrafiltartion and dialysed prior to a first chromatography. The dialysis buffer contained 50 mM Tris/HCl, 300 mM NaCl and 1 mM β-mercaptoethanol. Raw and still inactive chymase could be obtained by a chromatography on Ni—NTA using the same buffer and a gradient of imidazole to elute the protein from the column. Then the enzyme was activated by cleaving off the N-terminal 6×His tag with enterokinase. Thereby the mature guinea pig chymase with the correct N-terminus was formed. However, beside activation partial self digestion of the protein was also observed. The activated enzyme was twice chromatographed on Superdex 75, first in 50 mM Tris/HCl pH 8.0, 300 mM NaCl, 1 mM TCEP, 10% glycerol and then in 50 mM MES, 300 mM NaCl, 1 mM TCEP, 10% glycerol in a slightly acidic pH of 5.5 because chymase is described to be inactive at that pH (corresponds to the pH in mast cells, where active chymase is stored). Finally, as shown by SDS-PAGE >98% pure protein was obtained. The purified protein was monomeric as detected by analytical ultracentrifugation and highly active in a fluorometric assay.

### Example 10

### Chymase Activity Assays

The activity of guinea pig chymase is measured using familiar methods known to one of ordinary skill in the art, (Muramatsu et al., 2000, J. Biol. Chem. 275, page 5546; Uehara et al., 1999, Hypertension 35, page 57; Takai et al., 1997, Clin. Chim. Acta 265, p. 15), which are hereby incorporated by reference in their entirety.

### Example 10.1

### Assay for Chymase Inhibition

For the chymase a substrate was chosen containing the 4 amino acid peptide AAPF as a standard substrate for chymotrypsin like compounds (succinyl-Ala-Ala-Pro-Phe-[7-amino-4-methylcoumarin]; Lockhart B E, et al., “Recombinant human mast-cell chymase: an improved procedure for expression in Pichia pastoris and purification of the highly active enzyme.” Biotechnol Appl Biochem. published as immediate publication 26 May 2004 as manuscript BA20040074)). The peptide was synthesized with a purity of 95% from Bachem, Bubendorf, Switzerland). Chymase purified from human skin mast cells was obtained from Calbiochem (Merck Biosciences, San Diego, Calif., USA). The assay buffer was 0.15 M NaCl, 0.05M, Tris HCl, 0.05% CHAPS, 0.1 mg/ml Heparin (Heparin sodium, Sigma, porcine intestinal mucosa), 0.02mM AAPF-substrate, 1 nM Chymase at pH 7.4. The assay was performed in 96-well plates (Packard Optiplate), with a 0.05 ml volume at room temperature. Chymase activity was indicated by the initial rate of increase in fluorescence at 340/440 nm (excitation/emission) from free 7-amino-4-methylcoumarin released from the substrate. Inhibition of the activity by inhibitory compounds was read after 30 min pre-incubation with the chymase at room temperature in assay buffer without AAPF-substrate. The assay was then started by addition of the indicated concentration of AAPF-substrate.

TABLE 8
Inhibition of guinea pig chymase activity: (comparable to human chymase)
IC50 nM

| | Human | | |
|---|---|---|---|
| GP | Chymase | | |
| chymase | coli | | |
| 940 | 570 | ref cpd | Meiji Seika Kaisha (MSK) |
| | | | SF2809-V N-(2-{2-[(4-Hydroxy- |
| | | | 1-methyl-2-oxo-1,2-dihydro- |
| | | | quinolin-3-yl)-phenyl-methyl]- |
| | | | 1H-indol-3-yl}-ethyl)-acetamide |
| 345 | 590 | ref cpd | Toa Eiyo TY-51076 4- |
| | | | (5-Fluoro-3-methyl-benzothiophene-2- |
| | | | sulfonylamino)-3-methanesulfonyl- |
| | | | benzoic acid methyl ester |

1. A nucleic acid sequence comprising the sequence of Seq ID No. 1.

2. A polypeptide sequence comprising the sequence of Seq ID No. 2.

3. A nucleic acid sequence comprising a nucleic acid sequence that encodes the polypeptide sequence of claim 2.

4. An expression vector comprising the nucleic acid sequences of claim 3.

5. A host cell comprising the expression vector of claim 4.

6. A guinea pig with somatic cells and germ cells with a mutated chymase gene, wherein at least one allele is functionally interrupted through homologous recombination of the mutated chymase gene, wherein this functional interruption inhibits the expression of the functional chymase gene in the guinea pig cells.

7. A method for screening compounds that modulate the activity of chymase, comprising a) bringing a compound into contact with isolated polypeptide of claim 3; and b) determining the activity of guinea pig chymase; wherein a compound that inhibits or stimulates the activity of the guinea pig chymase is a compound that modulates the activity of the guinea pig chymase.

8. A method for identifying compounds that modulate the activity of chymase, comprising a) bringing a compound into contact with the host cell according to claim 5; and b) determining the activity of the guinea pig chymase that is expressed in the host cell; wherein a compound that inhibits or stimulates the activity of the guinea pig chymase is a compound that modulates the activity of the guinea pig chymase.

9. A method for identifying compounds that modulate the activity of chymase, comprising a) administering a compound to guinea pigs; and b) determining the activity of the guinea pig chymase; wherein a compound that inhibits or stimulates the activity of the guinea pig chymase is a compound that modulates the activity of the guinea pig chymase.
